(19) United States
(12) Patent Application Publication
(10) Pub. No.: US 2025/0288783 A1
(43) Pub. Date: Sep. 18, 2025

(54) MECHANISM FOR AXIALLY AND ROTATIONALY COUPLING A DILATOR AND SHEATH

(71) Applicant: Boston Scientific Scimed, Inc., Maple Grove, MN (US)

(72) Inventor: Jeffery D. Arnett, Louisa, VA (US)

(21) Appl. No.: 19/081,875

(22) Filed: Mar. 17, 2025

Related U.S. Application Data
(60) Provisional application No. 63/566,461, filed on Mar. 18, 2024.

(51) Int. Cl. A61M 29/00

(52) U.S. Cl. CPC A61M 29/00; A61M 2210/125

(57) ABSTRACT

A system for facilitating access to a patient's heart includes a sheath having a sheath body defining a lumen adapted to receive a dilator. The sheath body includes a proximal portion and a distal portion. A sheath hub is coupled to the proximal portion of the sheath. The sheath hub includes a passageway for receiving a portion of a dilator hub and a protrusion adjacent the passageway. The dilator has a dilator shaft defining a lumen to receive and support a puncturing device. The dilator shaft includes a proximal portion for manipulation by a user and a tapered distal portion for placement in or near the heart. A dilator hub is coupled to the proximal portion of the dilator shaft. The dilator hub includes a flange having an opening, the opening configured for receiving a portion of the sheath hub to inhibit relative rotation between the dilator and the sheath.

### CROSS REFERENCE TO RELATED APPLICATIONS

This application claims priority to U.S. Provisional Patent Application No. 63/566,461 entitled “MECHANISM AXIALLY AND ROTATIONALLY COUPLING A DILATOR AND SHEATH,” filed Mar. 18, 2024, which is incorporated herewith by reference in its entirety.

### TECHNICAL FIELD

The present invention relates generally to methods and devices usable to deliver a therapy to a patient. More specifically, the present invention is concerned with a system and method for delivering a therapy device to a heart.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein, and directed towards the heart. This device relies on the use of mechanical force to drive the sharp tip through the septum. Alternatively, radiofrequency perforation apparatuses have been developed, whereby the septal perforation is accomplished by the application of focused radiofrequency energy to the septal tissue via an electrode at the distal end of a relatively thin conductive probe.

Such perforation devices are often used in conjunction with a dilator to help support and guide the perforation device. Such dilators are often used in conjunction with a therapy sheath adapted to deliver a therapy to the patient.

### SUMMARY

Example 1 is a dilator for facilitating access to a patient's heart and for coupling with a sheath including a sheath hub. The dilator includes a dilator shaft defining a lumen adapted to receive and support a puncturing device. The dilator shaft includes a proximal portion for manipulation by a user and a distal portion for placement in or near the heart. A dilator hub is coupled to the proximal portion of the dilator shaft. The dilator hub includes a flange having an opening. The opening is configured for receiving a portion of the sheath hub to inhibit relative rotation between the dilator and the sheath.

Example 2 is the dilator of Example 1, wherein the dilator hub is directly coupled to the dilator shaft.

Example 3 is the dilator of Examples 1 or 2, wherein the sheath is a therapy sheath and the puncturing device is an RF puncturing device.

Example 4 is the dilator of any of Examples 1-3, further comprising an axial lock feature configured to create a resistance to an axial disengagement force, such that the dilator hub is secured axially within the sheath hub.

Example 5 is the dilator of any of Examples 1-4, wherein the axial lock feature includes a protrusion adapted to mate with a shoulder.

Example 6 is the dilator of Example 5, wherein the protrusion is annular.

Example 7 is the dilator of any of Examples 1-6, wherein the flange includes an asymmetric shape along a vertical axis and a symmetric shape along a horizontal axis.

Example 8 is the dilator of any of Examples 1-7, wherein the opening is aligned with the vertical axis or the horizontal axis.

Example 9 is the dilator of any of Examples 1-8, wherein the opening has an arc length in the range of 1 mm to 15 mm.

Example 10 is the dilator of any of Examples 1-9, wherein the opening has an arc angle measurement in the range of 20 degrees to 45 degrees.

Example 11 is the dilator of any of Examples 1-10, wherein a first half the flange is generally semicircular.

Example 12 is the dilator of any of Examples 1-11, wherein a second half of the flange is generally ellipse.

Example 13 is the dilator of any of Examples 11 or 12, wherein a first half of the flange has a first radius, and a second half of the flange has a second radius larger than the first radius, or the second radius is the same as the first radius, but eccentric.

Example 14 is the dilator of Example 13, wherein an opening is located in the second half of the flange.

Example 15 is the dilator of Example 14, wherein the opening has an arc length in the range of 1 mm to 15 mm and an arc angle measurement in the range of 20 degrees to 45 degrees.

Example 16 is a dilator for facilitating access to a patient's heart and for coupling with a sheath including a sheath hub. The dilator includes a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a flange having an opening. The opening is configured for receiving a portion of the sheath hub to inhibit relative rotation between the dilator and the sheath.

Example 17 is the dilator of Example 16, further comprising an axial lock feature configured to create a resistance to an axial disengagement force, such that the dilator hub is secured axially within the sheath hub.

Example 18 is the dilator of Example 17, wherein the axial lock feature includes an annular protrusion adapted to mate with a shoulder.

Example 19 is the dilator of Example 16, wherein the flange includes an asymmetric shape along a vertical axis and a symmetric shape along a horizontal axis.

Example 20 is the dilator of Example 16, wherein the opening is aligned with the vertical axis or the horizontal axis.

Example 21 is the dilator of Example 16, wherein the opening has an arc length in the range of 1 mm to 15 mm.

Example 22 is the dilator of Example 16, wherein the opening has an arc angle measurement in the range of 20 degrees to 45 degrees.

Example 23 is the dilator of Example 16, wherein a first half the flange is generally semicircular.

Example 24 is the dilator of Example 23, wherein a second half of the flange is generally ellipse.

Example 25 is the dilator of Example 16, wherein a first half of the flange has a first radius, and a second half of the flange has a second radius larger than the first radius, or the second radius is the same as the first radius, but eccentric.

Example 26 is the dilator of Example 25, wherein the opening is located in the second half of the flange.

Example 27 is a sheath for facilitating access to a patient's heart and for coupling with a dilator including a dilator hub. The sheath includes a sheath shaft defining a lumen adapted to receive and support a dilator. The sheath shaft includes a proximal portion for manipulation by a user and a distal opening through with the dilator can extend. A sheath hub is coupled to the proximal portion of the sheath shaft. The sheath hub includes a passageway for receiving a portion of the dilator hub and a protrusion adjacent the passageway. The protrusion is configured for contacting a flange of the dilator hub to inhibit relative rotation between the sheath and the dilator.

Example 28 is the sheath of Example 27, wherein the protrusion has an arc length in the range of 1 mm to 15 mm.

Example 29 is the sheath of Example 27, wherein the protrusion has an arc angle measurement in the range of 20 degrees to 45 degrees.

Example 30 is the sheath of Example 27, wherein the protrusion prevents incorrect insertion of the dilator into the sheath.

Example 31 is the sheath of Example 27, wherein the protrusion includes at least one angled surface.

Example 32 is a system for facilitating access to a patient's heart. The system includes a sheath having a sheath body defining a lumen adapted to receive a dilator. The sheath body includes a proximal portion and a distal portion. A sheath hub is coupled to the proximal portion of the sheath. The sheath hub includes a passageway for receiving a portion of a dilator hub and a protrusion adjacent the passageway. The system includes a dilator having a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a flange having an asymmetric shape along a vertical axis and a symmetric shape along a horizontal axis.

Example 33 is the system of Example 32, wherein the flange includes an opening aligned with the horizontal axis.

Example 34 is the system of Example 33, wherein the protrusion is configured to fit within the opening to inhibit relative rotation between the sheath and the dilator.

Example 35 is the system of Example 33, wherein the opening has an arc length in the range of 1 mm to 15 mm and an arc angle measurement in the range of 20 degrees to 45 degrees.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the invention.

FIG. 2 is a perspective view of a dilator with a dilator hub being inserted into a sheath hub, according to embodiments of the invention.

FIG. 3 is a perspective view of the dilator hub of FIG. 2 mating with the sheath hub of FIG. 2, according to embodiments of the invention.

FIG. 4A and FIG. 4B are perspective views of the dilator and sheath hub of FIG. 2 in incorrect orientations, according to embodiments of the invention.

FIG. 5 is an end view of the sheath hub of FIG. 2, according to embodiments of the invention.

FIG. 6 is an end view of the dilator hub of FIG. 2, according to embodiments of the invention.

FIG. 7A is a cross-sectional view of the dilator hub of FIG. 2 partially mated with the sheath hub of FIG. 2, according to embodiments of the invention.

FIG. 7B is a cross-sectional view of the dilator hub of FIG. 2 fully mated with the sheath hub of FIG. 2, according to embodiments of the invention.

While the invention is amenable to various modifications and alternative forms, specific embodiments have been shown by way of example in the drawings and are described in detail below. The intention, however, is not to limit the invention to the embodiments described. On the contrary, the invention is intended to cover all modifications, equivalents, and alternatives falling within the scope of the invention as defined by the appended claims.

### DETAILED DESCRIPTION

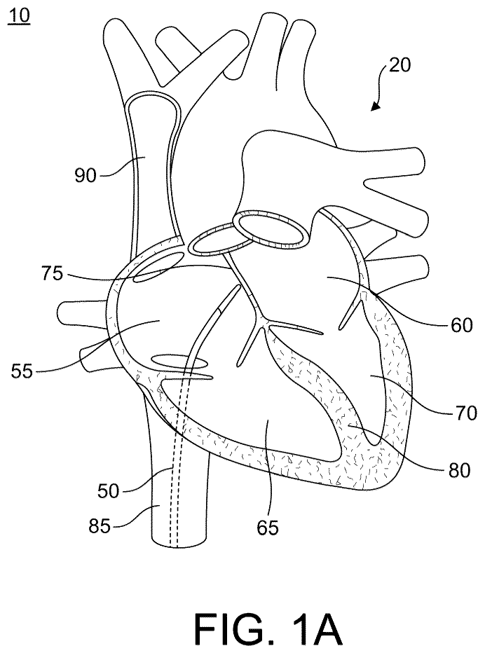

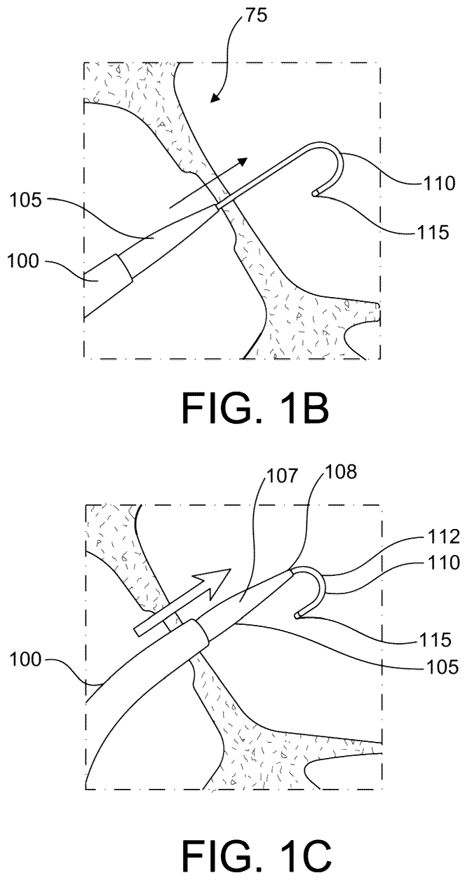

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a perforation device (e.g., a radiofrequency (RF) perforation device) 110 having distal end portion 112 terminating in a tip electrode 115. As shown, in the assembled use state illustrated in FIGS. 1A-1C, the RF puncturing device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the SVC 90, the guidewire is removed from the body, and the sheath 100 and the dilator 105 are retracted so that their distal ends are positioned in the right atrium 55. The RF puncturing device 110 described can then be introduced into the dilator 105, and advanced toward the heart 20. In various embodiments, the guidewire and the RF puncturing device are the same component, so an exchange is not necessary.

Subsequently, the user may position the distal end of the dilator 105 against the atrial septum 75, which can be done under imaging guidance. The RF puncturing device 110 is then positioned such that electrode 115 is aligned with or protruding slightly from the distal end of the dilator 105. The dilator 105 and the RF puncturing device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF puncturing device 110 is usable to facilitate positioning of the tip electrode 115 at the desired target site.

With the tip electrode 115 and dilator 105 positioned at the target site, energy is delivered from an energy source, e.g., an RF generator, through the RF puncturing device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF puncturing device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF puncturing device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF puncturing device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF puncturing devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF puncturing device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF puncturing device 110 into the left atrium 60. The anchoring function of the pre-formed distal end portion 112 inhibits unintended retraction of the distal end portion 112, and corresponding loss of access to the perforated site on the atrial septum 75, during such withdrawal.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

In certain embodiments, catheters, therapy devices and sheaths can be deployed through the sheath 100, after it is successfully deployed into the desired heart chamber (e.g., the left atrium). In other embodiments, the therapy device (e.g., mapping catheter, therapy sheath, medical device, etc.) is part of the sheath 100, creating a therapy sheath.

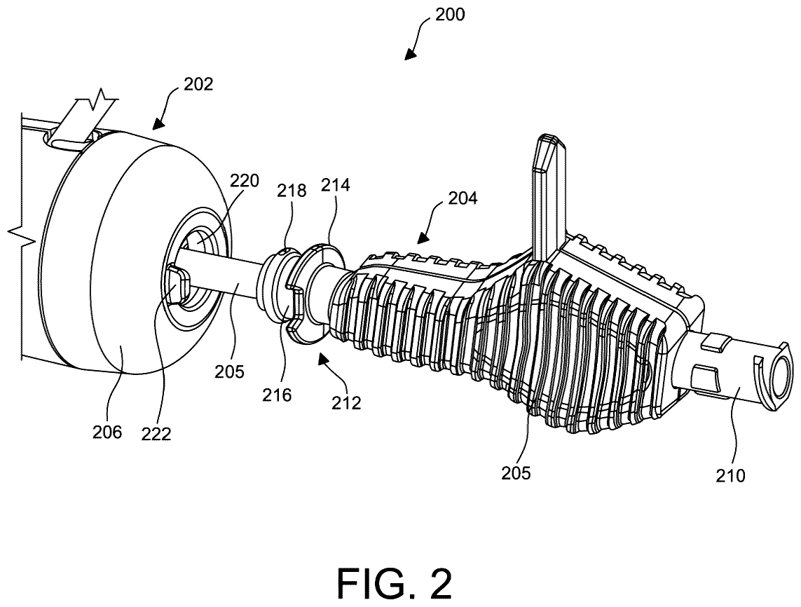

FIG. 2 is a perspective view of a system 200 for facilitating access to a patient's heart. The system includes a dilator 204 with a dilator hub 212 configured for insertion into a sheath 202 having a sheath hub 206, according to embodiments of the invention. As shown, the dilator 204 is partially inserted into a lumen of the sheath 202. The dilator 205 includes a handle 208, a dilator hub 212, a proximal connector 210, and a dilator shaft 205. The sheath 202 includes a sheath hub 206 and a sheath shaft (not shown) defining a lumen adapted to receive and support the dilator shaft 205. The sheath shaft includes a proximal portion for manipulation by a user and a distal opening through with the dilator can extend as discussed above in FIGS. 1A-1C. The sheath hub 206 is coupled to the proximal portion of the sheath shaft. The sheath hub 206 can be directly coupled or indirectly coupled to the sheath shaft. The sheath hub 206 includes a passageway 220 for receiving a portion of the dilator hub and a protrusion 222 adjacent the passageway 220. The protrusion 222 is configured for contacting a flange 214 of the dilator hub 212 to inhibit relative rotation between the sheath 202 and the dilator 204, such that when a user rotates either the sheath 202 or the dilator 204 they rotate together as a single unit.

The dilator 204 includes a dilator shaft 205 defining a lumen adapted to receive and support a puncturing device. A puncturing device can be inserted into the dilator 204 using the proximal connector 210. The proximal connector 210 is also configured to connect to a fluid introducing apparatus such as a syringe or other container for delivering fluid, such as a contrast medium, through the dilator shaft 205. The puncturing device can include a mechanical perforation device, for example a guidewire having a needle tip, or an RF puncturing device. The dilator shaft 205 includes a proximal portion for manipulation by a user and a distal portion for placement in or near the heart. A dilator hub 212 is coupled to the proximal portion of the dilator shaft 205. The dilator hub 212 can be directly coupled or indirectly coupled to the dilator shaft 205. The dilator hub 212 includes a flange 214 having an asymmetric shape along a vertical axis and a symmetric shape along a horizontal axis. The flange 214 includes an opening 216 that is configured to receive the protrusion 222. When the protrusion 222 is received in the opening 216, relative rotation between the dilator 204 and the sheath 202 is inhibited or prevented. This creates a rotational lock between the two components. This allows a user to simultaneously rotate the dilator 204 and sheath 202 together.

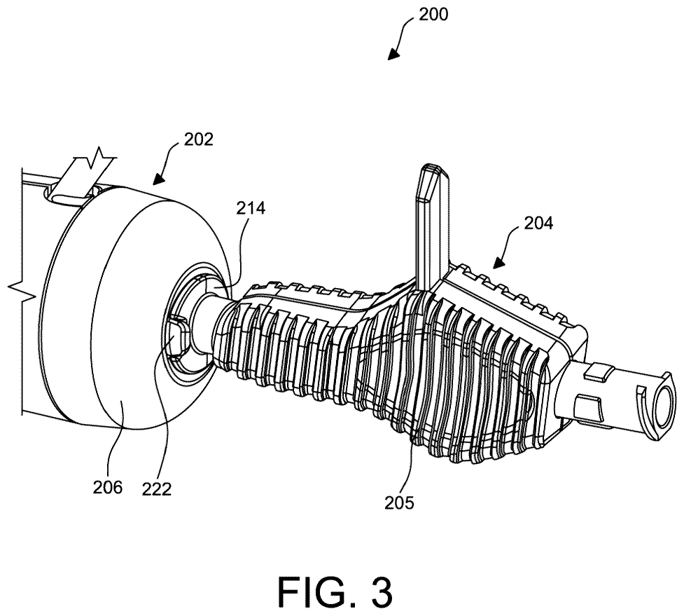

FIG. 3 is a perspective view of the dilator hub 212 of FIG. 2 properly seated with and fully inserted into the sheath hub 206 of FIG. 2, according to embodiments of the invention. As shown in FIG. 3, the dilator hub 212 is inserted into the passageway 220 until the flange 214 contacts a surface of the sheath hub 206. In this orientation, relative rotation of the dilator 204 with respect to the sheath 202 is prevented. The protrusion 222 must be aligned with the opening 216 in order for the flange 214 to fully abut the sheath hub 206 and the protrusion 222 to be received in the opening 216.

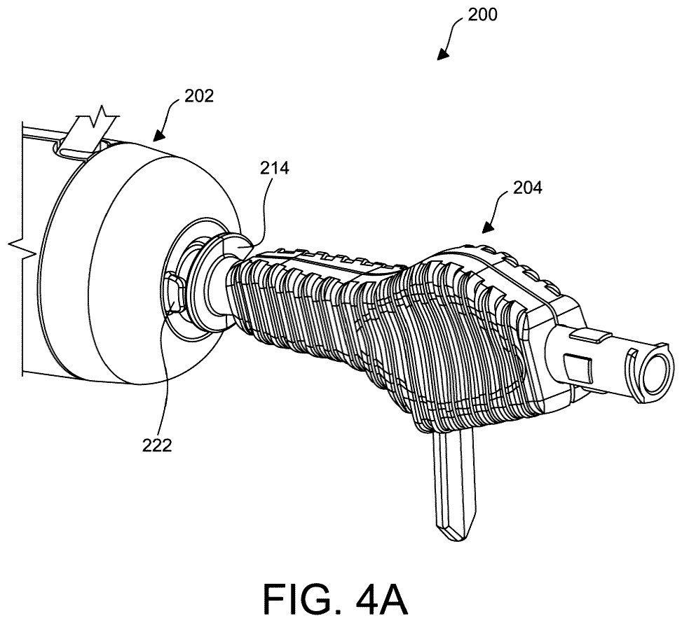

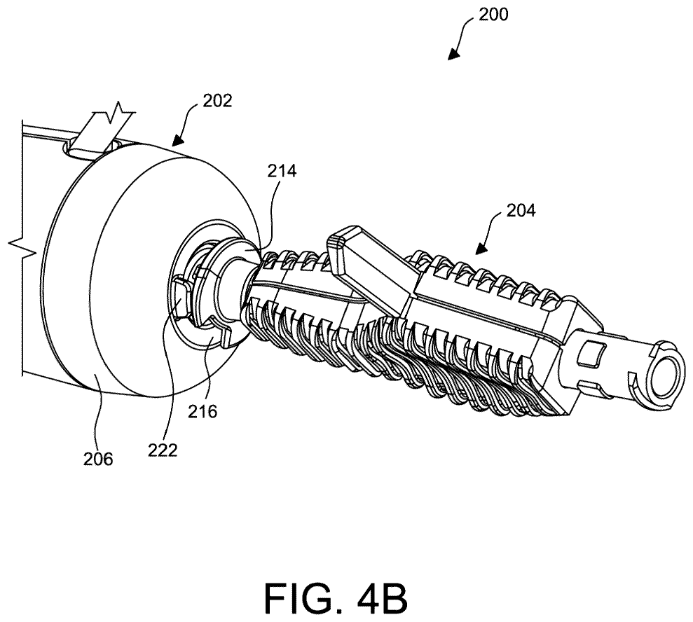

FIG. 4A and FIG. 4B are perspective views of the dilator 204 and sheath hub 206 of FIG. 2 in incorrect orientations. In FIG. 4A, the dilator 204 is rotated substantially 180 degrees from being correctly inserted into the sheath 202. As illustrated, the flange 214 abuts the protrusion 222. This prevents complete insertion of the dilator hub 212 into the sheath hub 206 and provides a user with tactile feedback that the dilator 204 and sheath 202 are not properly aligned. Likewise, in FIG. 4B, the dilator 204 does not have proper alignment with the sheath 202. As such, the dilator hub 212 is prevented from being received properly in the sheath hub 206 by interaction of the flange 214 and the protrusion 222.

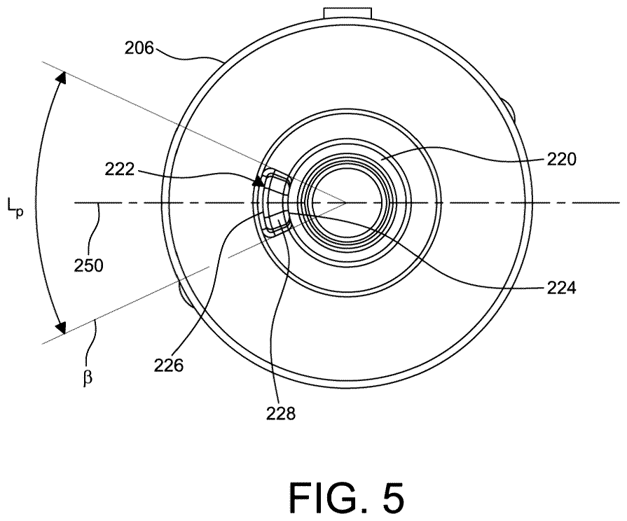

FIG. 5 is an end view of the sheath hub 206 of FIG. 2, according to embodiments of the invention. The sheath hub 206 includes a passageway 220 for receiving a portion of the dilator hub 212. A protrusion 222 is adjacent the passageway 220. The protrusion 222 is aligned with a horizontal axis passing through the center of the sheath hub 206. The protrusion 220 is configured for contacting the flange 214 of the dilator hub to inhibit relative rotation between the sheath and the dilator, by being located within the opening 216. The protrusion 222 has an arc length Lₚ in the range of 1 mm to 15 mm. The protrusion 222 has an arc angle measurement β in the range of 20 degrees to 45 degrees. The arc length Lₚ of the protrusion 222 is minimized such that the protrusion 222 does not interfere with a user gripping the dilator 204, but also provides enough strength to prevent the protrusion 222 from breaking off.

The protrusion 222 includes an inner surface 224 closest to the opening 216 and an outer surface 226 opposite the inner surface 226. An edge 228 extends between the inner surface 224 and the outer surface 226. A height of the inner surface 224 and the outer surface 226 can be different such that the edge 228 is angled relative to a longitudinal axis of the sheath hub 206 that passes through the opening 216.

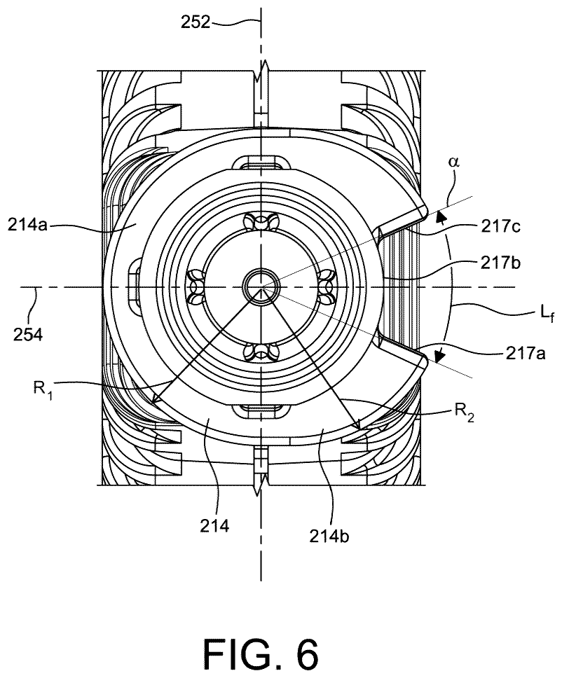

FIG. 6 is an end view of the dilator hub 212 of FIG. 2, according to embodiments of the invention. As shown in FIG. 6, the flange 214 of the dilator hub 212 has an asymmetric shape along a vertical axis 252 and a symmetric shape along a horizontal axis 254. The vertical axis 252 and the horizontal axis 254 pass through a longitudinal axis of the dilator 204. A first half 214a the flange 214 is generally semicircular. The first half 214a of the flange 214 includes a radius R1. A second half 214b of the flange 214 is generally ellipse. In some embodiments, a first half 214a of the flange 214 has a first radius R1, and a second half 214b of the flange 214 has a second radius R2 larger than the first radius R1. In some embodiments, the second half 214b of the flange 214 has the same radius R1 as the first half 214a but is eccentric. In some embodiments, the flange 214 does not have an asymmetric shape along the vertical axis 252, but instead has a consistent radius.

As illustrated in FIG. 6, the opening 216 is located in the second half 214b of the flange 214. The opening 216 is aligned with an axis orthogonal to the longitudinal axis of the dilator. In one embodiment, the opening 216 is aligned with the horizontal axis 254. The opening has an arc length Li in the range of 1 mm to 15 mm. The opening 216 has an arc angle measurement α in the range of 20 degrees to 45 degrees. The opening 216 partially surrounds the protrusion 222 when mated with the protrusion 222. The outer surface 226 of the protrusion 222 is not encompassed by the opening 216. The opening 216 includes surfaces that interact with the edge 228 of the protrusion.

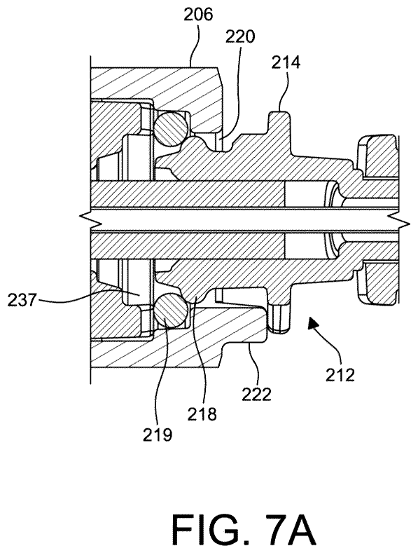

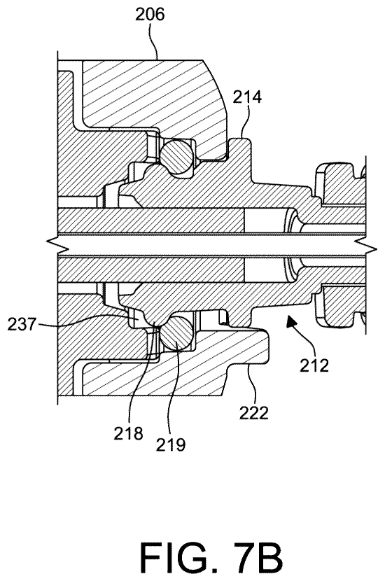

FIG. 7A is a cross-sectional view of the dilator hub 212 of FIG. 2 partially mated with the sheath hub 206 of FIG. 2 and FIG. 7B is a cross-sectional view of the dilator hub 212 fully mated with the sheath hub 206, according to embodiments of the invention. The distal end of the dilator hub 212 includes an axial lock feature, which includes a protrusion 218. In some embodiments, the protrusion 218 is an annular protrusion located on an outer circumference of the dilator hub 212. This annular protrusion 218 is adapted to engage with a corresponding shoulder 219 within the sheath hub 206. The interaction between the protrusion 218 and the shoulder 219 creates a resistance to an axial disengagement force, such that the dilator hub 212 is secured axially within the sheath hub 206. In one aspect, the shoulder 219 takes the form of an elastic and deformable o-ring. Upon insertion of the dilator hub 212 into the sheath hub 206, the annular protrusion 218 deforms the o-ring and enters into a locking chamber 237 as illustrated in FIG. 7B. The annular protrusion 218 remains within the locking chamber 237, unable to move proximally past the shoulder 219, in order to axial lock the dilator hub 212 to the sheath hub 206. In one aspect, the locking chamber 237 is configured to allow slight axial movement of the annular protrusion 218 by providing space between the shoulder 219 and the annular protrusion 218. In another aspect, the locking chamber 237 is configured to securely hold the annular protrusion 218, wherein the annular protrusion 218 is in contract with the shoulder 219. The dilator hub 212 and the sheath hub 206 are configured such that when the annular protrusion 218 is positioned within the locking chamber 237, the flange 214 abuts the sheath hub 206 and the protrusion 222 is located within the opening 216 of the flange 214, so as to resist or prevent relative rotation as discussed above.

In order to remove the dilator hub 212 from the sheath hub 206, a disengagement force can be created by interaction of the angled surface of the edge 228 with surfaces 217a, 217b, 217c. The angled surface of the edge 228 and surfaces 217a, 217b, 217c may have slightly different angles or the same angle (but not oriented horizontally), such that upon application of a torque to the dilator hub 212, an axial disengagement force is generated. The disengagement force (having an axial force component) causes an axial motion of the dilator hub 212, which disengages the dilator hub 212 from the sheath hub 206. When the disengagement force becomes sufficient to overcome an axial lock force formed by an interaction with the annular protrusion 218 and the shoulder 219, relative motion in both axial and rotational directions occurs, allowing removal of the dilator hub 212 from the sheath hub 206. By changing the angles of these surfaces, the amount of torque and the degree of rotation required for disengagement may be adjusted.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A dilator for facilitating access to a patient's heart and for coupling with a sheath including a sheath hub, the dilator comprising:
a dilator shaft defining a lumen adapted to receive and support a puncturing device, the dilator shaft includes a proximal portion for manipulation by a user and a tapered distal portion for placement in or near the heart; and
a dilator hub coupled to the proximal portion of the dilator shaft, the dilator hub including a flange having an opening, the opening configured for receiving a portion of the sheath hub to inhibit relative rotation between the dilator and the sheath.

2. The dilator of claim 1, further comprising an axial lock feature configured to create a resistance to an axial disengagement force, such that the dilator hub is secured axially within the sheath hub.

3. The dilator of claim 2, wherein the axial lock feature includes an annular protrusion adapted to mate with a shoulder.

4. The dilator of claim 2, wherein the flange includes an asymmetric shape along a vertical axis and a symmetric shape along a horizontal axis.

5. The dilator of claim 1, wherein the opening is aligned with the vertical axis or the horizontal axis.

6. The dilator of claim 1, wherein the opening has an arc length in the range of 1 mm to 15 mm.

7. The dilator of claim 1, wherein the opening has an arc angle measurement in the range of 20 degrees to 45 degrees.

8. The dilator of claim 1, wherein a first half the flange is generally semicircular.

9. The dilator of claim 8, wherein a second half of the flange is generally ellipse.

10. The dilator of claim 1, wherein a first half of the flange has a first radius, and a second half of the flange has a second radius larger than the first radius, or the second radius is the same as the first radius, but eccentric.

11. The dilator of claim 10, wherein the opening is located in the second half of the flange.

12. A sheath for facilitating access to a patient's heart and for coupling with a dilator including a dilator hub, the sheath comprising:
a sheath shaft defining a lumen adapted to receive and support a dilator, the sheath shaft includes a proximal portion for manipulation by a user and a distal opening through with the dilator can extend; and
a sheath hub coupled to the proximal portion of the sheath shaft, the sheath hub including a passageway for receiving a portion of the dilator hub and a protrusion adjacent the passageway, the protrusion configured for contacting a flange of the dilator hub to inhibit relative rotation between the sheath and the dilator.

13. The sheath of claim 12, wherein the protrusion has an arc length in the range of 1 mm to 15 mm.

14. The sheath of claim 12, wherein the protrusion has an arc angle measurement in the range of 20 degrees to 45 degrees.

15. The sheath of claim 12, wherein the protrusion prevents incorrect insertion of the dilator into the sheath.

16. The sheath of claim 12, wherein the protrusion includes at least one angled surface.

17. A system for facilitating access to a patient's heart, the system comprising:
a sheath having a sheath body defining a lumen adapted to receive a dilator, the sheath body including a proximal portion and a distal portion;
a sheath hub coupled to the proximal portion of the sheath, the sheath hub including a passageway for receiving a portion of a dilator hub and a protrusion adjacent the passageway;
a dilator having a dilator shaft defining a lumen adapted to receive and support a puncturing device, the dilator shaft including a proximal portion for manipulation by a user and a tapered distal portion for placement in or near the heart; and
a dilator hub coupled to the proximal portion of the dilator shaft, the dilator hub including a flange having an asymmetric shape along a vertical axis and a symmetric shape along a horizontal axis.

18. The system of claim 17, wherein the flange includes an opening aligned with the horizontal axis.

19. The system of claim 18, wherein the protrusion is configured to fit within the opening to inhibit relative rotation between the sheath and the dilator.

20. The system of claim 18, wherein the opening has an arc length in the range of 1 mm to 15 mm and an arc angle measurement in the range of 20 degrees to 45 degrees.
